(19) United States
(12) Patent Application Publication
(10) Pub. No.: US 2004/0030152 A1
(43) Pub. Date: Feb. 12, 2004

(54) Magnesium anodisation system and methods

(76) Inventor: John Arnold Macculloch, Auckland (NZ)

Correspondence Address: LOWE HAUPTMAN GILMAN AND BERNER, LLP, 1700 DIAGONAL ROAD, SUITE 300 /310, ALEXANDRIA, VA, 22314, US

(21) Appl. No.: 10/398,415

(22) PCT Filed: Oct. 5, 2001
(86) PCT No.: PCT/NZ01/00215

(30) Foreign Application Priority Data
Oct. 5, 2000 (NZ) 507363
Feb. 9, 2001 (NZ) 509895

(51) Int. Cl. C07D 207/00

(52) U.S. Cl. 548/400

(57) ABSTRACT

This invention relates to a method of anodising magnesium material. The method includes anodising the magnesium material whilst it is immersed in an aqueous electrolyte solution having a pH above 9, and in the presence of a phosphate. The phosphate is preferably an ortho-phosphate, and the solution also preferably includes a buffering agent such as tetra-borate to maintain the pH of the solution above 9. There are also described method steps prior to anodising. One step describes a cathodic cleaning of the magnesium material, prior to it being anodised. Another step describes a pre-treatment which preferably includes one or more substeps, namely a cleaning step and/or an etching step and/or a surface activation step.

### TECHNICAL FIELD

This invention relates to magnesium anodising systems and methods. Throughout this specification, the terms “magnesium”, “magnesium metal” and “magnesium material”, may be used interchangeably, and are all to be understood to refer to or include magnesium metal and/or magnesium alloy(s) and/or mixtures thereof, and/or any articles or compounds comprising or including magnesium.

### BACKGROUND ART

Magnesium is a very light, yet strong metal and is finding increasing acceptance for metal die castings, particularly where weight savings are desired. In addition, its property of shielding electromagnetic radiation is causing it to be of interest as a replacement for plastics in applications such as computers and mobile telephones. However, it is a reactive metal and corrosion, whether general or by galvanic effects, is a major problem.

A number of methods for applying a protective anodic oxide film on magnesium material have been available. These have sought to imitate the well established processes available for coating aluminium and its alloys, however achieving the same result on magnesium articles has been extremely difficult. In part this is due to the fact that the oxide formed from a given volume of magnesium metal occupies less space than the original metal and thus any film of the oxide formed on the surface is subject to tensile stress and before a significant layer can be built up, it cracks and spalls away from the substrate.

The anodisation of aluminium and its alloys is often conducted in sulphuric acid in which the oxide layer formed is slightly soluble. As the film builds outwards from the metal substrate, its rate of build decreases, so ultimately there is an equilibrium point at which the rate of dissolution is equal to that of further film growth. The dissolution of the film causes the formation of pores through which the ionic migration necessary to the electrochemical oxidation of the metal takes place. Without these pores only very thin films would be possible. After the electrochemical oxidation process is complete, the pores are sealed. Sealing of anodised aluminium can be achieved with hot water or simple inorganic chemical solutions.

Clearly an analogous process involving magnesium would attempt to simulate these features. However, because of the tendency of the forming film to crack and break due to the imposed tensile stresses, there are complications. Also, the use of an acidic solution to anodise magnesium is fraught with serious difficulties as magnesium is rapidly attacked by most common acids. Therefore, anodisation of magnesium should preferably take place in alkaline solutions.

One method of anodising magnesium relies on this property to create a rough, very porous layer which may form a base for paint or other surface coatings to be applied afterwards. Commonly, such an anodic film may be formed in an electrolyte of high pH, containing alkali hydroxides. The process proceeds by means of sparking, which sparking forms a sintered ceramic oxide film as the metal substrate is coated.

However, the forming of a sintered ceramic oxide film, through sparking, is not always desirable as the film is often brittle, uneven, and/or lacks uniformity.

A number of proprietary methods for anodisation of magnesium exist which seek to avoid this problem and hence create a stronger and/or more uniform film.

In PCT/NZ96/00016 (WO 96/28591) (Barton) there is disclosed a viable procedure for anodising magnesium or magnesium alloys. It involves anodising the material in an ammonia containing electrolyte solution. The presence of some phosphate compounds in the solution is also disclosed. Enhancements of such a Barton procedure are disclosed in PCT/NZ98/00040 (W098/42892) (MacCulloch et al).

For environmental reasons arising from the emanation of ammonia and also taking into account potential problems associated with the disposal of ammonia-containing electrolytes and process washings, a process is desirable beyond those aforesaid where no ammonia or ammonium salts are present in the electrolyte. However, the absence of ammonium compounds imposes difficulties in the functioning of the process in the areas of anodic polarisation, repeatability and film quality.

It is one object of the present invention to provide, as an alternative to the processes of WO 96/28591 or WO 98/42892, a process (together with related apparatus and products) in which no (or negligible amounts of) ammonia or ammonium salts are present in the electrolyte, whilst also producing a substantially strong, and/or uniform, and/or even film.

It is a further object of the present invention to address the foregoing problems or at least to provide the public with a useful choice.

Further aspects and advantages of the present invention will become apparent from the ensuing description that is given by way of example only.

### DISCLOSURE OF INVENTION

According to one aspect of the present invention there is provided a method of anodising magnesium which includes anodising the magnesium material whilst it is immersed in an aqueous electrolyte solution having a pH above 9, and in the presence of a phosphate.

According to another aspect of the present invention there is provided a method, substantially as described above, wherein the phosphate includes an ortho-phosphate.

According to another aspect of the present invention there is provided a method, substantially as described above, wherein the phosphate includes a pyro-phosphate.

According to another aspect of the present invention there is provided a method, substantially as described above, wherein the phosphate is an alkali metal phosphate.

According to another aspect of the present invention there is provided a method, substantially as described above, wherein the solution includes at least one buffering agent to maintain the pH of the solution above 9.

According to another aspect of the present invention there is provided a method, substantially as described above, wherein the solution includes a tetra-borate.

According to another aspect of the present invention there is provided a method, substantially as described above, wherein the tetra-borate is an alkali metal tetra-borate.

According to another aspect of the present invention there is provided a method, substantially as described above, wherein the alkali metal tetra-borate is sodium tetra-borate.

According to another aspect of the present invention there is provided a method, substantially as described above, wherein the solution includes an alkali metal hydroxide.

According to another aspect of the present invention there is provided a method, substantially as described above, wherein the solution includes magnesium sulphate as a source of magnesium cations.

According to another aspect of the present invention there is provided a method, substantially as described above, wherein the pH is between 10.2-11.0.

According to another aspect of the present invention there is provided a method, substantially as described above, wherein the temperature of the electrolyte solution is between 20° C.-60° C.

According to another aspect of the present invention there is provided a method, substantially as described above, wherein the temperature of the electrolyte solution is approximately 40° C.

According to another aspect of the present invention there is provided a method, substantially as described above, wherein the anodising of the magnesium material includes passing a current through the solution and/or through the magnesium material for one to five minutes.

According to a further aspect of the present invention there is provided a method, substantially as described above, wherein the current is between 50-500 Amp/m² of the magnesium material.

The phosphate in the electrolyte may preferably be in the form of phosphate ions. The phosphate ions may preferably be sourced from a water soluble phosphate salt which has been dissolved in the electrolyte solution.

Preferably, the solution may only contain an ortho-phosphate. Any suitable ortho-phosphate, or salt thereof may be utilised, although an alkali metal phosphate such as sodium ortho-phosphate may be particularly suitable.

Similarly, if the solution also or alternatively contains a pyro-phosphate, an alkali metal phosphate such as sodium pyro-phosphate may be particularly suitable.

The pH may be maintained above 9 (which is the magnesium/magnesium hydroxide equilibrium pH in water), and preferably in the range from 10-12. A particularly suitable pH range may be 10.2-11.0.

Preferably, the pH may be adjusted to the preferred range using a base, which may preferably have identical cations to the cations of the ortho and/or pyro-phosphate and/or the tetra-borate. For example, if the phosphate ions are provided by sodium ortho-phosphate, and/or the tetra-borate is provided by sodium tetra-borate, then the base may preferably be provided by sodium hydroxide, namely NaOH. Other known bases may however be used as required or desired.

The electrolyte may preferably include a source of magnesium cations. The source of magnesium cations may be forthcoming from one or both of the liquid which may be used as the anodising electrolyte, and/or the addition or inclusion in the electrolyte of a magnesium salt. For example, for a one bath system, the magnesium ions from the added salt may be in the nature of a “starter”, and thereafter from the anodising as a continuing supply. Preferably the magnesium salt may be magnesium sulphate.

Preferably, the cations in the solution, save for those of magnesium, may be largely those of an alkali metal, and preferably those of sodium.

The anodic reaction may take place in a vessel in which the magnesium material to be anodised is connected to an electrically-conductive rack and immersed in the electrolyte. Generally, the rack may be coated in plastic except for small contact areas where it forms an electrical connection to the magnesium material being anodised. Where the rack is composed of a material that will passivate under the electrical conditions of the anodising process, it may not be necessary to coat the rack with an insulator, but it may be desirable to do so for improved efficiency.

In general it may be advantageous for the vessel containing the electrolyte and the magnesium material to be anodised to be made of insulating plastic, provided that electrically conductive counter-electrodes are inserted in the tank, most commonly in the sides. It is desirable that these be inert chemically, preferably of stainless steel, for example type 316. Although it is possible to use counter-electrodes composed of alternative substances, for example, aluminium, this is undesirable since in another modification of the process, as described below, a reverse polarity voltage may be applied to the article resulting in a brief, anodic polarisation. Stainless steel has the advantage of being inert under these conditions whereas aluminium would anodise, preventing the proper functioning of the standard cycle.

The electrolyte may be operable over a broad temperature range, from around zero to its boiling point, but the process may operate optimally over a range 20-60° C., and more particularly at approximately 40° C.

The voltage applied to the electrolyte may preferably be direct current. The output produced by a rectified three phase power supply, comprising a voltage of constant polarity fluctuating by approximately 5% is suitable, as is smoothed DC. Modified waveforms, for instance, pulsed or superimposed AC voltages may also be employed although these result in different film thickness and other characteristics than that normally obtained from direct current anodisation.

When an anodic voltage is first applied to the magnesium material to be anodised the electrical resistance is low but this progressively increases as an insulating anodic film forms on the surface. The result is an increasing voltage when the anodising current is held constant. The process is preferably controlled by means of a constant current, preferably in the range 50 A/m² to 500 A/m² and optimally around 200 A/m². When operated at 200 A/m², the imposed voltage may be expected to reach 200 volts after two to three minutes, and for a commercially-useful coating, the voltage may reach an ultimate limit of 230 to 270 volts. Very thin films, suitable for some applications may be achieved using lower voltages. The film continues to build if the voltage is held constant on attaining a certain limit, for example, 220 volts, and as this takes place, the current dwindles.

Since power supplies vary in their characteristics and the ultimate voltage achieved for an equivalent film thickness is highly dependent on aspects such as ripple percentage, the presence or absence of pulses and other electrical characteristics, the voltages stated above are given by way of example only. The process is operable over a broad range of voltages and current densities.

It is desirable that the anodising electrolyte has efficient circulation both for reasons of maintaining uniform electrolyte composition and heat removal. Many suitable circulation means or apparatus are known for ensuring circulation within a bath or electrolyte and need not be described further herein.

The methods disclosed by Barton and MacCulloch are optimally conducted at temperatures lower than 10° C., thereby requiring the use of compressive refrigeration to remove waste heat from the process solution. This entails considerable capital expenditure and additional energy costs. For the purposes of the present invention, a cooling tower is sufficient for commercial production. The result is a significant saving.

In still a further aspect the present invention consists in an electrolyte composition suitable for use in an anodising electrolytic cell for a magnesium material including, in aqueous solution at a pH above 9, the following:

1) a pyro or ortho-phosphate (preferably ortho-phosphate) of from about 10 to 30g/l (preferably about 20g/l) when expressed as NaH₂(PO₄).

2) a tetra-borate of from about 15 to 50g/l (preferably about 30g/1) when expressed as Na₂B₄O₇ and (optionally)

3) a source of magnesium cations (e.g. a salt).

Preferably the composition includes both NaH₂(PO₄) (preferably about 20g/l) and Na₂B_{4O}₇ (preferably about 30g/l).

Preferably a base or bases such as an alkali metal hydroxide may be present to provide a pH of 9 or above, and the base is preferably NaOH. Preferably NaOH base may be present in a range of from about 10 to 20g/l and preferably about 15g/l.

Preferably the salt may be magnesium sulphate and it is preferably present in about 0.5 to 2 g/l and most preferably about 1g/l.

The buffering agent, such as a tetra-borate, not only assists in controlling the pH during reaction but also tends to lower the resistance of the electrolyte and helps in depolarising the anode.

In still another aspect the invention consists in a method of anodising a magnesium material including:

immersing said magnesium material in an electrolyte as an anode;

providing a cathode in or for said electrolyte; and

passing a current through said electrolyte;

and wherein the electrolyte, possessing a pH greater than about 9, comprises in aqueous solution phosphate ions, a compatible buffering agent, and, optionally, free magnesium cations.

Preferably said phosphate ions are of ortho-phosphate.

Preferably a compatible tetra-borate such as sodium tetra-borate buffers the pH.

According to a further aspect of the present invention there is provided a method, substantially as described above, wherein the anodising of the magnesium material follows a cathodic cleaning of the magnesium material.

According to a further aspect of the present invention there is provided a method, substantially as described above, wherein the cathodic cleaning takes place in the same solution (or electrolytic cell) substantially as described above with respect to the anodising of the magnesium material.

According to a further aspect of the present invention there is provided a method, substantially as described above, wherein the cathodic cleaning step includes passing a current through the solution and/or through the magnesium material for a time which is substantially the same as that for the current used in respect of the anodising of the magnesium material.

According to a further aspect of the present invention there is provided a method, substantially as described above, wherein the strength of the current is substantially the same as that used for the anodising of the magnesium material.

According to a further aspect of the present invention there is provided a method, substantially as described above, wherein the anodising step immediately follows the cathodic cleaning step by reversing the polarity or direction of the current.

In a further aspect, the present invention consists in a method of anodising a magnesium material, said method comprising:

A) cathodically cleaning the magnesium material in a solution containing a source of pyro-phosphate ions, and

B) anodising the cathodically cleaned magnesium material in an electrolyte solution having a source of ortho-phosphate ions, the electrolyte solution during such anodisation being buffered to a pH above 9 or otherwise being maintained above 9.

Preferably the solution of step A includes a tetra-borate (eg; sodium tetra-borate).

Preferably the electrolyte of the anodising electrolytic cell is substantially that of the cathodic cleaning cell i.e. the magnesium material is not moved between its cathodic cleaning and its anodising. Preferably at least some of the magnesium cations present in the electrolyte of the anodising electrolytic cell have been removed during the previous anodising of magnesium material. Preferably however a salt of magnesium is also present in the electrolyte of the anodising cell e.g. preferably as magnesium sulphate.

Preferably, the temperature of the solution during the cathodic cleaning step may be substantially the same as that during the anodising step, namely, preferably in the range of 20° C.-60° C., and most preferably approximately 40° C.

The cathodic cleaning step includes passing a current through the solution and/or through the magnesium material. Any suitable time which enables the magnesium material to be cathodically cleaned (that is, adequately cleaned so that the subsequent anodising process will be efficient) may be utilised. A suitable time may be between 1-5 minutes, with approximately 2 minutes being particularly suitable.

Similarly, any suitable current strength may be utilised as required. A current of between 100- 500 Amp/m² may be suitable, and 200 Amp/m² being particularly suitable.

Preferably, the anodising step may immediately follow the cathodic cleaning step by simply reversing the polarity or direction of the current.

In still a further aspect this present invention consists in apparatus for anodising magnesium material where the magnesium material can first be operated in the electrolyte solution as a cathode and thereafter (preferably without disconnection and reconnection, e.g. preferably by simple or automated switching) can thereafter be operated as an anode to achieve the anodisation thereof in the presence of free magnesium ions, at least some of which have been generated into solution whilst the magnesium or magnesium alloy(s) has been operated as an anode, said electrolyte having a pH of 9 or above and having a phosphate species present.

According to another aspect of the present invention there is provided a method of anodising magnesium material, substantially as described above, wherein the anodising of the magnesium material follows a pre-treatment designed to prepare the magnesium material for anodisation.

According to another aspect of the present invention there is provided a method, substantially as described above, wherein the pre-treatment includes one or more of the following substeps:

(a) a cleaning step,

(b) an etching step,

(c) a surface activation step.

According to another aspect of the present invention there is provided a method, substantially as described above, wherein there is a cleaning step before and after the etching step.

According to another aspect of the present invention there is provided a method, substantially as described above, wherein the cleaning step includes an immersion of the magnesium material into a solution containing caustic soda.

According to another aspect of the present invention there is provided a method, substantially as described above, wherein the etching step includes an immersion of the magnesium material into a solution containing at least one acid.

According to another aspect of the present invention there is provided a method, substantially as described above, wherein the acid is nitric acid or phosphoric acid.

According to another aspect of the present invention there is provided a method, substantially as described above, wherein the etching step includes an immersion of the magnesium material into a solution containing DEOXALUME™.

According to another aspect of the present invention there is provided a method, substantially as described above, wherein the surface activation step includes an immersion of the magnesium material into a solution containing a source of fluoride ions.

According to another aspect of the present invention there is provided a method, substantially as described above, wherein the surface activation step includes an immersion of the magnesium material into a solution containing a source of fluoride ions and an acid.

According to another aspect of the present invention there is provided a method, substantially as described above, wherein the surface activation step includes an immersion of the magnesium material into a solution containing potassium fluoride and nitric acid or phosphoric acid.

According to another aspect of the present invention there is provided a method, substantially as described above, wherein the surface activation step includes an immersion of the magnesium material into a solution containing ammonium bifluoride.

According to another aspect of the present invention there is provided a method, substantially as described above, wherein the surface activation step includes an immersion of the magnesium material into a solution containing DEOXALUME™.

According to another aspect of the present invention there is provided a method, substantially as described above, wherein between each substep there is optionally a rinsing step.

Generally speaking, the methods described in Barton and MacCulloch do not usually require a thorough cleaning of the magnesium metal prior to the anodisation process. This is because the electrolytes described in Barton and MacCulloch contain ammonia, which is very effective in an electrolyte solution for the purposes of anodising magnesium material regardless (to a certain extent) of the cleanliness of the magnesium material prior to anodising.

We have found that by pre-treating the magnesium material, prior to anodisation, we are able to achieve a virtually equivalent result as regards the quality of the anodic film formed (as regards uniformity, strength and evenness etc) as the processes described in Barton and MacCulloch, but without requiring ammonia to be present in the electrolyte. This is desirable given that we are no longer faced with the environmental/health problems associated with using and disposing of ammonia containing electrolytes, as described previously.

Generally speaking, surface cleaning and preparation of metal substrates for an electro chemical process is a complex field and the pre-treatments presented below are therefore given by way of example only. Moreover, in some situations where components are heavily soiled, for example with die lubricants, or have surface corrosion, special cleaning steps additional or alternative to those listed herein may be required. Alternatively, good quality components with clean surfaces may require fewer or less rigorous cleaning steps.

The at least one pre-treatment steps described above as (a), (b), (c) may be undertaken in any order and/or may be repeated as required or as desired, or as dictated by the condition of the magnesium material to be pre-treated and subsequently anodised. Furthermore, and again depending upon the condition of the magnesium material, only one or two (or three) of the pre-treatment substeps may be utilised.

Preferably, the cleaning step may be followed by the etching step, and subsequently followed by the surface activation step. Alternatively, or additionally, there may be provided an additional cleaning step after the etching step, but prior to the surface activation step. The cleaning step may involve the immersion of the magnesium material into an appropriate cleaning solution.

Preferably, the cleaning step may involve the immersion of the magnesium material into a solution which includes caustic soda. Any suitable concentration may be utilised as required or as desired, or as dictated by the condition of the magnesium material to be cleaned.

Preferably, the caustic soda may include sodium hydroxide at a concentration of between 10- 50% w/v. A concentration of approximately 30% w/v may be particularly suitable.

The magnesium material may be immersed in the cleaning solution for any length of time, as required or as desired, or as dictated by the condition of the magnesium material. Preferably, the immersion time may be between 2-12 minutes, with approximately 5 minutes being particularly suitable.

The caustic soda solution may be at any suitable temperature, as required or as desired, or as dictated by the condition of the magnesium material. Preferably, the solution may be at a temperature of between 50-95° C., with a range of 70-85° C. being particularly suitable.

Preferably, after the cleaning step the magnesium material may be rinsed, and preferably with water. De-ionised water may be particularly suitable.

The etching step may preferably include the immersion of the magnesium material into a solution containing at least one acid. Any suitable acid or acids may be utilised as required or as desired. Examples include nitric acid and phosphoric acid.

Any suitable concentrations of acid may be utilised as required or as desired. For example, if the acid used is nitric acid, it may preferably be of a concentration of approximately 0.4-0.8M, with a particularly suitable range being 0.5-.0.6M.

Altenatively, the magnesium material may be immersed into a solution containing DEOXALUME ™, which is a proprietary product manufactured by Henkel Corporation. If DEOXALUME™ is used, it may preferably be diluted to, approximately a 10% concentration.

The etching step serves to remove surface layers of the magnesium material which assists in the anodisation process.

The magnesium material may be immersed in the etching solution for any length of time, as required or as desired or as dictated by the state of the magnesium material. For example, if phosphoric acid or nitric acid were to be used a time of approximately 30 seconds to 4 minutes may be suitable. If DEOXALUME™ is used, a time of approximately 10-30 seconds may be suitable.

Similarly, the temperature of the etching solution may be in the range of 10-80° C., with a range of approximately 20-40° C. being particularly suitable.

Preferably, the magnesium material may be rinsed after the etching step, and preferably with water. De-ionised water may be particularly suitable.

Preferably, a further cleaning step, substantially as described previously, may be undertaken after the etching step, and preferably a further rinsing of the magnesium material, for example with de-ionised water, may follow the second cleaning step.

The surface activation step may preferably follow the (second) cleaning step and/or the etching step.

One purpose of the surface activation step is to provide the magnesium material with a thin film of magnesium fluoride. It is found that the anodisation process is greatly enhanced, and the quality of the anodic film increased if the surface activation step, including the depositing of a thin film of MgF₂, is carried out prior to the anodisation process.

It appears that the thin film of magnesium fluoride suppresses the tendency for the “corrosion” reaction to take place (as compared to the “coating” reaction which occurs during anodising). Namely, it appears that the magnesium fluoride film mediates the conditions in the electric double layer between the metal substrate and the bulk solution so that the alternative “corrosion” reaction is largely suppressed. The corrosion reaction is where magnesium metal becomes magnesium ions, which subsequently grab water and become Mg²+_{aq}. These aqueous magnesium ions may then grab on to hydroxide ions or phosphate ions thus forming insoluble compounds which may show up s defects in the anodised magnesium material.

In one embodiment, the surface activation step may include immersing the magnesium material into a solution containing DEOXALUME™, the solution being of substantially the same concentration and at the same temperature substantially as described previously in respect of the etching step.

Preferably however the surface activation step may include immersing the magnesium material into a solution containing a source of fluoride ions. Preferably, the solution may also contain an acid, for example phosphoric acid or nitric acid. Preferably, a sufficient amount of acid is added to adjust the pH of the solution to between 2-3.

Preferably, the source of the fluoride ions may be potassium fluoride.

Preferably, the magnesium material may be immersed in a solution for 1030 seconds (although this time would of course change depending upon the condition of the magnesium material. Preferably, the temperature of the solution may be between 20-50° C., with the range of 20-40° C. being particularly suitable. The concentration of potassium fluoride may be adjusted as required or as desired, although a strength of approximately 5-10 g/l may be particularly suitable.

Alternatively, the surface activation may involve immersing the magnesium material into solutions containing ammonium bifluoride, with the concentration of the ammonium bifluoride preferably being in the range of 0.5-2%w/v, and preferably at a temperature range of approximately 25-55° C. The magnesium material may be immersed into the solution for any suitable length of time. A time of between 2-8 minutes may be suitable, with a time of approximately 5 minutes being particularly suitable.

Preferably, after the surface activation step the magnesium material is again rinsed, preferably with water. De-ionised water may be particularly suitable.

The scope of the present invention is also to be understood to include apparatus which may be utilised for anodising the magnesium material, substantially as described herein. Further more, it is also within the scope of this invention to include a magnesium material product, having been anodised according to the methods substantially as described herein.

According to a further aspect of the present invention there is also provided an electrolyte composition suitable for use in an anodising electrolytic cell for magnesium material, including, in an aqueous solution at a pH above 9, the following:

(a) a phosphate,

(b) a tetra-borate,

(c) a source of magnesium cations,

(d) at least one base.

This invention may also be said broadly to consist in the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.

Wherein, in the foregoing statements of invention or in the following description, MgSO₄, Na₂B₄O₇ and/or Na₂H₂PO₄ are described or detailed, it will be appreciated that hydrated forms of these compounds are also contemplated. In particular (and vice versa). In particular, the forms MgSO₄.7H₂O, Na₂B₄O₇.5 H₂O and Na₂H₂PO₄.2H₂O may also be used. It will bee appreciated that where specific concentrations of the anhydrous compounds are listed, the molar concentrations will need to be adjusted accordingly for the hydrated forms (and vice versa).

Preferred forms of the present invention will now be described with reference to the accompanying drawings in which

### BRIEF DESCRIPTION OF DRAWINGS

FIG. 1A shows an electrolyte bath in which a magnesium material is acting as a cathode thereby freeing magnesium cations into the electrolyte whilst also tending to clean or etch the surface, and

FIG. 1B shows the reversed polarity situation where the magnesium material is anodic and can form an anodised layer thereon.

FIG. 2 shows a flow diagram illustrating an appropriate pretreatment path according to another aspect of the present invention.

### BEST MODES FOR CARRYING OUT THE INVENTION

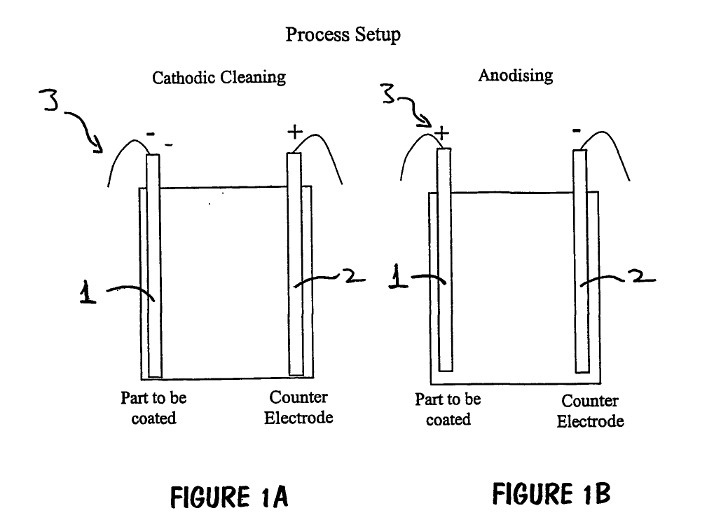

In the electrolytic cell of FIGS. 1A and 1B, a stainless steel electrode 2 is used as the second electrode, the magnesium material being the other electrode 1.

The electrolyte of the present invention (in the embodiment where the magnesium material is to be operated first as a cathode and then as an anode) preferably consists of or at least includes:

a) An alkali metal phosphate, either pyro or ortho with the latter being preferable (e.g. NaH₂(PO₄)).

b) An alkali metal borate (preferably sodium tetra-borate Na₂B₄O₇).

c) An alkali metal hydroxide (e.g. NaOH).

d) Magnesium sulphate (e.g. MgSO₄).

Preferably both the cathodic cleaning and anodisation process should be performed at a current density of 100 to 500 A/m², where the area referred to is the apparent geometric surface area of the magnesium alloy work piece.

The cell process voltages can be delineated as:

A) Cleaning—from 0.5 to 15 volts

Preferably—10 volts

B) Anodising—from 0 to 400 volts

Preferably—200 volts

Note that these voltages are end of process voltages and in the case of B, it is a function of the desired film thickness.

A typical electrolyte composition is;

TABLE 1

| | |
|---|---|
| NaH₂(PO₄).2H₂O | 20 g/l |
| Na₂B₄O₇.5H₂O | 30 g/l |
| NaOH | 15 g/l |
| MgSO₄.5H₂O | 1 g/l |

The first two components are preferably dissolved in water at 25 to 40° C. The NaOH is added as a pre-dissolved solution with stirring, until the pH is brought in the interval 10.2 to 11.0. The MgSO4 is finally added in similar fashion.

The electrolyte is useable over the temperature range 5 to 85° C. but preferably it is used in the range 30 to 70° C.

The article to be coated 1 is placed in the electrolytic cell 3, which contains electrolyte and a stainless steel counter-electrode 2, usually equal or greater than the area of the article.

The cell 3 is connected to a suitable DC power supply that has a means of controlling current, voltage and output polarity.

The cell 3 is initially polarised so that the article to be coated 1 is made a cathode (i.e. negative) the counter electrode 2 is thus connected to the positive terminal of the supply.

Current (usually at 200 A/m² of the article area) is passed for up to 1 minute. After this time the polarity applied to the cell 3 is reversed so that the article 1 is now connected to the positive end of the supply and preferably the same current is passed for a time that will deposit the coating to the desired thickness.

During the process, the applied voltage will rise with coating thickness and for a given current density will eventually reach a limiting value dictated by electrode composition, temperature and the geometry of the cell.

At this point the reactions occurring at the electrodes are those corresponding to the electrolytic decomposition of the water i.e. the evolution of hydrogen at the cathode and oxygen at the anode. The latter stages of the process may be accompanied by a visible plasma discharge on the anode, which may not be desirable.

After coating the article 1 is disconnected and removed from the cell 3 and thoroughly washed in de-ionised water to remove soluble salts. It may then be hot air dried.

The combination of the tetra-borate ion and an operating pH greater than 9 (preferably a pH>10) has been able to resolve problems encountered previously in the absence of ammonia.

The use of pyro-phosphate alone as the source of phosphorous is not as reliable as ortho-phosphate as pyro-phosphate compounds can revert to the ortho form at elevated temperatures and the pyro-phosphates also exert a certain degree of complexing action on the magnesium that can lead to film dissolution as a competing reaction.

The presence of relatively large amounts of sodium ions has two disadvantages when compared to the ammonium ion process.

Firstly, the post anodisation washing process must be more thorough as Na⁺is detrimental to the corrosion properties of the coating and are highly conductive.

Secondly the maximum coating thickness available by this new process can be lower than the previous process as the breakdown voltage at the anode is lower, thus shunting process current into electrolyte decomposition rather than into film deposition in the latter stages of the process.

However, for films up to about 8 μm in thickness, this new process appears to be more energy efficient. This is mainly because the average voltage of the cell is lower and the coating can be somewhat rougher in nature thus giving an apparently greater thickness for a given mass of deposited material.

As the anodising bath can operate at relatively higher temperatures than one containing ammonia compressive refrigeration is not required therefore additional energy savings can be made. Baths with no ammonia have been successfully operated at up to 90° C. although the electrical efficiency may suffer at such high temperatures.

Coatings produced using any one of the anodising electrolytes with the properties described can be produced more economically and are free of some environmental difficulties (associated with ammonia). Given the limitations described they are perfectly adequate for a large number of applications.

Process Example:

A. Electrolyte Solution:

| | |
|---|---|
| Composition | as in Table 1 |
| Temperature | 40° C. |

B. Process:

1) The Work piece was an alloy plate of area 0.02 m² processed at a current density of 200 A/m².

2) The counter electrode was a stainless steel plate of similar area.

3) The work piece made the cathode and a current of 3.0 A DC was passed for a period of 30 sec with an end magnitude passed in this direction for a period of 6 minutes.

4) Immediately the polarity was reversed, making the work piece the anode with a current of the same magnitude passed in this direction for a period of 6 minutes.

5) The voltage at the end of this time was 212 volts.

6) After anodising, the sample was removed, washed and hot air dried

7) The thickness of the buff coloured film was measured with an eddy current meter and was found to be 5.5 μm thick.

As an alternative to the cathodic treating of the magnesium material prior to anodising, the magnesium material may instead be pre-treated. The pre-treatment preferably includes the following steps, namely a cleaning step, an etching step, and a surface activation step.

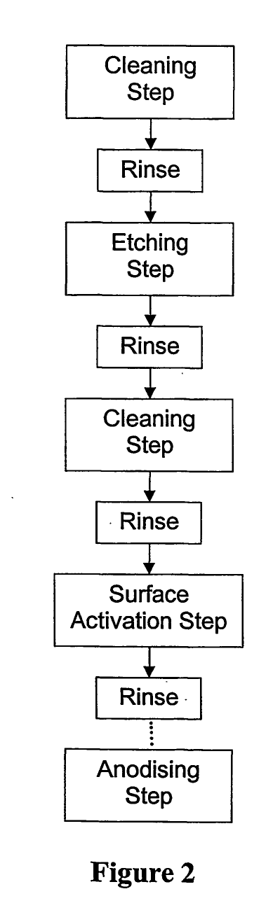

Preferably, the magnesium material is first subjected to a cleaning step followed by the etching step, followed by a further cleaning step, and followed lastly by a surface activation step. Preferably, in between each of the steps as just described, there is a rinsing step involving the rinsing of the magnesium material with de-ionised water. This pretreatment process is summarised in FIG. 2.

Some examples of best modes for carrying out the invention, utilising the pre-treatment step, are described below:

1. An electrolyte was prepared as follows:

Sodium dihydrogen orthophosphate (NaH₂PO₄.2H₂O)—6 g/l Sodium tetra-borate (Na₂B₄07.5H₂O)—30 g/l Sodium hydroxide (NaOIH)—approx 10 g/l

The phosphate salt was dissolved in deionised water, and the borate added slowly at a temperature of around 40° C. Sodium tetra-borate pentahydrate, as used in this example, is quite slow to dissolve as there is a tendency for the formation of large, slow-to-dissolve crystals. The pH was then adjusted upwards to 11.0 by adding sodium hydroxide solution.

Pre-treatment steps were as follows:

(i) 5 minutes in 25% NaOH solution at 80° C.

(ii) Rinsing with de-ionised water.

(iii) 2 minutes in 3.5% nitric acid at ambient temperature.

(iv) Rinsing with de-ionised water.

(v) 5 minutes in 25% NaOH solution at 80° C.

(vi) Rinsing with de-ionised water.

(vii) 5 minutes in 0.03M ammonium bifluoride at 40° C.

(viii) Rinsing with de-ionised water.

Anodising was then performed in the electrolyte described above at 200 A/m², with the voltage starting from zero and rising to around 230 volts before the process was terminated. The anodic film formed was smooth and uniform.

2. An electrolyte was prepared as follows:

5 Sodium dihydrogen orthophosphate (NaH₂PO₄.2H₂O)—12 g/l

Sodium tetra-borate (Na₂B₄07.5H₂O)—15 g/l

Sodium hydroxide (NaOH)—approx 15 g/l

The electrolyte was prepared as for the previous example. Pre-treatments were as for the previous example. The anodising was conducted at 200 A/m², with the voltage stating from zero and reaching about 230 volts. A smooth, uniform film, similar to that described in example #1 above resulted.

In any of the examples given above, after coating, the magnesium material may be disconnected and removed from the electrolytic cell, thoroughly washed in de-ionised water to remove soluble salt, and then hot air dried.

We have found that utilising the methods, substantially as described above, results in an anodic coating on the magnesium material which is of substantially the same quality as those produced previously using ammonia However, the present invention has the advantage of not using ammonia, which does away with the handling, ventilation and/or health problems associated with same.

Aspects of the present invention have been described by way of example only and it should be appreciated that modifications and additions may be made thereto without departing from the scope thereof as defined in the appended claims.

The claims defining the invention are:

1. A method of anodising magnesium material which includes anodising the magnesium material whilst it is immersed in an aqueous electrolyte solution having a pH above 9, and in the presence of a phosphate.

2. A method as claimed in claim 1 wherein the phosphate includes an ortho-phosphate.

3. A method as claimed in claim 1 or claim 2 wherein the phosphate includes a pyro-phosphate.

4. A method as claimed in any one of claims 1 to 3 wherein the phosphate is an alkali metal phosphate.

5. A method as claimed in any one of claims 1 to 4 wherein the solution includes at least one buffering agent to maintain the pH of the solution above 9.

6. A method as claimed in any one of claims 1 to 5 wherein the solution includes a tetra-borate.

7. A method as claimed in claim 6 wherein the tetra-borate is an alkali metal tetra-borate.

8. A method as claimed in claim 7 wherein the alkali metal tetra-borate is sodium tetra-borate.

9. A method as claimed in any one of claims 1 to 8 wherein the solution includes an alkali metal hydroxide.

10. A method as claimed in any one of claims 1 to 9 wherein the solution includes magnesium sulphate as a source of magnesium cations.

11. A method as claimed in any one of claims 1 to 10 wherein the pH is between 10.2-11.0.

12. A method as claimed in any one of claims 1 to 11 wherein the temperature of the electrolyte solution is between 20° C.-60° C.

13. A method as claimed in claim 12 wherein the temperature of the electrolyte solution is approximately 40° C.

14. A method as claimed in any one of claims 1 to 13 wherein the anodising of the magnesium material includes passing a current through the solution and/or through the magnesium material for one to five minutes.

15. A method as claimed in claim 14, wherein the current is between 50-500 Amp/m² of the magnesium material.

16. A method of anodising magnesium material, as claimed in any one claims 1 to 15, wherein the anodising of the magnesium material follows a cathodic cleaning of the magnesium material.

17. A method as claimed in claim 16 wherein the cathodic cleaning takes place in the same solution (or electrolytic cell) as that used for the anodising of the magnesium material.

18. A method as claimed in claim 16 or claim 17, wherein the cathodic cleaning step includes passing a current through the solution and/or through the magnesium material for a time which is substantially the same as that for the current used in respect of the anodising of the magnesium material.

19. A method as claimed in claim 18, wherein the strength of the current is substantially the same as that used for the anodising of the magnesium material.

20. A method as claimed in any one of claims 16 to 19 wherein the anodising step immediately follows the cathodic cleaning step by reversing the polarity or direction of the current.

21. A method of anodising magnesium material, as claimed in any one of claims 1 to 15, wherein the anodising of the magnesium material follows a pre-treatment designed to prepare the magnesium material for anodisation.

22. A method as claimed in claim 21 wherein the pretreatment includes one or more of the following substeps:
(a) a cleaning step,
(b) an etching step,
(c) a surface activation step.

23. A method as claimed in claim 22 wherein there is a cleaning step before and after the etching step.

24. A method as claimed in claim 22 or claim 23 wherein the cleaning step includes an immersion of the magnesium material into a solution containing caustic soda.

25. A method as claimed in any one of claims 22 to 24 wherein the etching step includes an immersion of the magnesium material into a solution containing at least one acid.

26. A method as claimed in claim 25 wherein the acid is nitric acid or phosphoric acid.

27. A method as claimed in any one of claims 22 to 24, wherein the etching step includes an immersion of the magnesium material into a solution containing DEOXALUME™.

28. A method as claimed in any one of claims 22 to 27 wherein the surface activation step includes an immersion of the magnesium material into a solution containing a source of fluoride ions.

29. A method as claimed in any one of claims 22 to 28 wherein the surface activation step includes an immersion of the magnesium material into a solution containing potassium fluoride and nitric acid or phosphoric acid.

30. A method as claimed in any one of claims 22 to 28 wherein the surface activation step includes an immersion of the magnesium material into a solution containing ammonium bifluoride.

31. A method as claimed in any one of claims 22 to 28 wherein the surface activation step includes an immersion of the magnesium material into a solution containing DEOXALUME™.

32. A method as claimed in any one of claims 22 to 31 wherein between each substep there is optionally a rinsing step.

33. A method of anodising magnesium material which includes the following steps:
(a) cathodically cleaning the magnesium material,
(b) subsequently anodising the magnesium material while it is immersed in an aqueous electrolyte solution having a pH above 9, and in the presence of a phosphate.

34. A method of anodising magnesium material which includes the following steps:
(a) pre-treating the magnesium material in order to prepare the magnesium material for anodisation,
(b) subsequently anodising the magnesium material while it is immersed in an aqueous electrolyte solution having a pH above 9, and in the presence of a phosphate.

35. A method as claimed in claim 34 wherein the pre-treatment includes one or more of the following substeps:
(a) a cleaning step,
(b) an etching step,
(c) a surface activation step.

36. An electrolyte composition for use in anodising magnesium material as claimed in any one claims 1 to 13.

37. An electrolyte composition suitable for use in an anodising electrolytic cell for magnesium material, including, in an aqueous solution at a pH above 9, the following:
(a) a phosphate,
(b) a tetra-borate,
(c) a source of magnesium cations,
(d) at least one base.

38. A method of anodising magnesium material which includes the following steps:
(a) immersing the magnesium material in an electrolyte as an anode,
(b) providing a cathode in or for the electrolyte,
(c) passing a current through the electrolyte,
wherein the electrolyte has a pH greater than 9, and includes an aqueous a phosphate.

39. A method as claimed in claim 38, wherein the electrolyte also includes tetra-borate, and free magnesium cations.

40. Apparatus for anodising the magnesium material as claimed in any one of claims 1 to 39.

41. Magnesium material anodised according to the methods as claimed in any one of claims 1 to 39.

42. A method of anodising magnesium material, substantially as herein described and with reference to the accompanying drawings.
